(19) United States
(12) Patent Application Publication
(10) Pub. No.: US 2007/0086756 A1
(43) Pub. Date: Apr. 19, 2007

(54) Static generator of compressed hot air for delivery to cyclically operated utilizing appliances

(76) Inventor: Renato Rimondi, Bazzano (IT)

Correspondence Address: STITES & HARBISON PLLC, 1199 NORTH FAIRFAX STREET, SUITE 900, ALEXANDRIA, VA, 22314, US

(21) Appl. No.: 10/579,751

(22) PCT Filed: Dec. 2, 2004
(86) PCT No.: PCT/EP04/53246

§ 371 Date: May 18, 2006

(30) Foreign Application Priority Data
Dec. 18, 2003 (IT) BO2003A000760

(51) Int. Cl. A61L 9/03; A01G 13/06; A01M 13/00; A01M 19/00; A61B 16/00

(52) U.S. Cl. 392/386

(57) ABSTRACT

The electrical resistance is housed in a composite body made of a material which is a good heat conductor, externally insulated so as not to leak heat into the external environment and machined in any way so that it comprises internally at least two separate and offset channels (4, 5) having identical dimensional characteristics and having essentially equal surface areas of contact with the resistance and essentially equal coefficients of heat exchange and that are provided with respective inlet ports (112, 113) and with respective outlet ports (118, 119), the inlet ports being connectable as required through switching-valve means (16) to the source (17) supplying the compressed air for heating, while the two discharge ports of the said channels are connected, one to the utilizing appliance (21), for example to the heating unit, to the sealing unit, to the cutting station or to another cyclically operated utilizing appliance and the other discharge port is connected to an exhaust duct (19) that ends in any position remote from the said utilizing appliance, the whole in such a way that by supplying one or other of the said channels with the stream of compressed ambient air, the compressed hot air produced by the generator can be sent to the utilizing appliance or to the said exhaust duct while maintaining unchanged heat exchange conditions between the resistance and the stream of air whatever path the air flows, owing to the structural identity and to the uniformly distributed offset locations of the said channels.

In machines for packaging fresh food products in thermoplastic film, it is known practice to employ generators of hot air which is then supplied for example to sealers joining together two superimposed edges of film, for instance making so-called longitudinal seals, or the hot air may be supplied to heating units for improving the physical properties of the film during the formation of the packaging or even on the finished packaging. A packaging machine which has these requirements is disclosed for example in WO 03051715 and WO 2004009450, and is used for packaging fresh products placed in trays, in a stretch film optionally having a barrier effect. This machine comprises hot-air generators that use electrical resistances controlled by thermostats and pressure sensors, through which the air or other gas is forced so that it is heated as it comes into contact with the said resistance and is channelled towards the utilizing appliances characterized by being operated cyclically. If the hot air is required for making longitudinal seals on the seal packaging, the same hot air produced by the static generator is channelled through a flexible tube to a sealing head mounted on means which normally keep it close to the film during the active sealing phase but which move it away from the film during those phases of the cycle in which the packaging stops temporarily for longitudinal stretching. At the appropriate moment as the sealer is moved away from the packaging film, means such as for example a jet of cold air, or other means, intervene to deflect the stream of hot air directed at the film. With this approach the electrical resistance is continuously struck by a stream of air and functions at steady load so whenever the sealer is raised it is always in the optimal working condition. On the other hand, this approach has not been found satisfactory due to the presence of the said means for moving the sealer back and forth, the action of which can deteriorate over time due to wear because it can pick up non-tolerable frictions and inertias and because these means may be struck accidentally by the operator when changing the forming mandrel for the tubular winding of the film, as the characteristics of the products to be packaged vary.

If the hot air produced by the generator is to be supplied to stations where the packaging film is thermally conditioned, valve means are installed downstream of the generator to direct the stream of hot air to the said packaging stations when the latter are to be active, whereas they switch the stream of hot air in another direction when these stations are to be inactive; all in such a way that, once again, as in the application to the sealer, the electrical resistance of the generator is in a constant stream of air and is not damaged by becoming overheated. For this approach there are problems of balancing the flow rates in the two different situations of sending hot air to the utilizing appliances or to the exhaust and there are problems of reliability over time with the switching system that acts on the stream of hot air.

Another problem encountered with the known solutions discussed above is the fact that the electrical resistance is in direct contact with the airflow to be heated. This means that small droplets of water or other impurities contained in the air inevitably damage the resistance and put all the utilizing appliances connected to the air generator out of service.

It is an object of the invention to solve these and other technical problems with the following idea for a solution. The resistance is housed in a composite body made of a material which is a good heat conductor, externally insulated so as not to leak heat into the environment and machined in such a way that it comprises at least two separate internal channels having identical dimensional characteristics and having essentially equal but offset and uniformly distributed surface areas of contact with the resistance and equal coefficients of heat exchange and that are provided with respective inlet ports and with respective outlet ports. The inlet ports of the said channels may be connected as required through switching-valve means to the source supplying the compressed air for heating which may for example be at room temperature. The discharge ports of the said channels are connected, one to the utilizing appliance of the machine, for example to the sealer or to the thermal conditioning station, or to a cutting station (see later), while the other is connected to an exhaust duct that ends in any suitable location of the packaging machine. It will be obvious that, by supplying one or other of the said channels through valve means which operate on the cold air stream and are therefore highly reliable, it is possible to deliver the hot air produced by the generator to the utilizing appliance or to the said exhaust duct, with the confidence that the heat exchange conditions between the resistance and the stream of air remain unchanged independently of the path followed by the air itself, owing to the structural identity and equal coefficient of heat exchange of the said channels. In a preferred embodiment of the invention, the said internal channels of the hot-air generator, which prevent direct contact between the air and the electrical resistance, are shaped as adjacent cylindrical helices, like the threads of a two-start screw.

Other features of the invention, and the resulting advantages, will be made clearer in the course of the following description of a preferred embodiment of the invention, illustrated purely by way of non-restrictive example, in the figures of the attached sheet of drawings, in which:

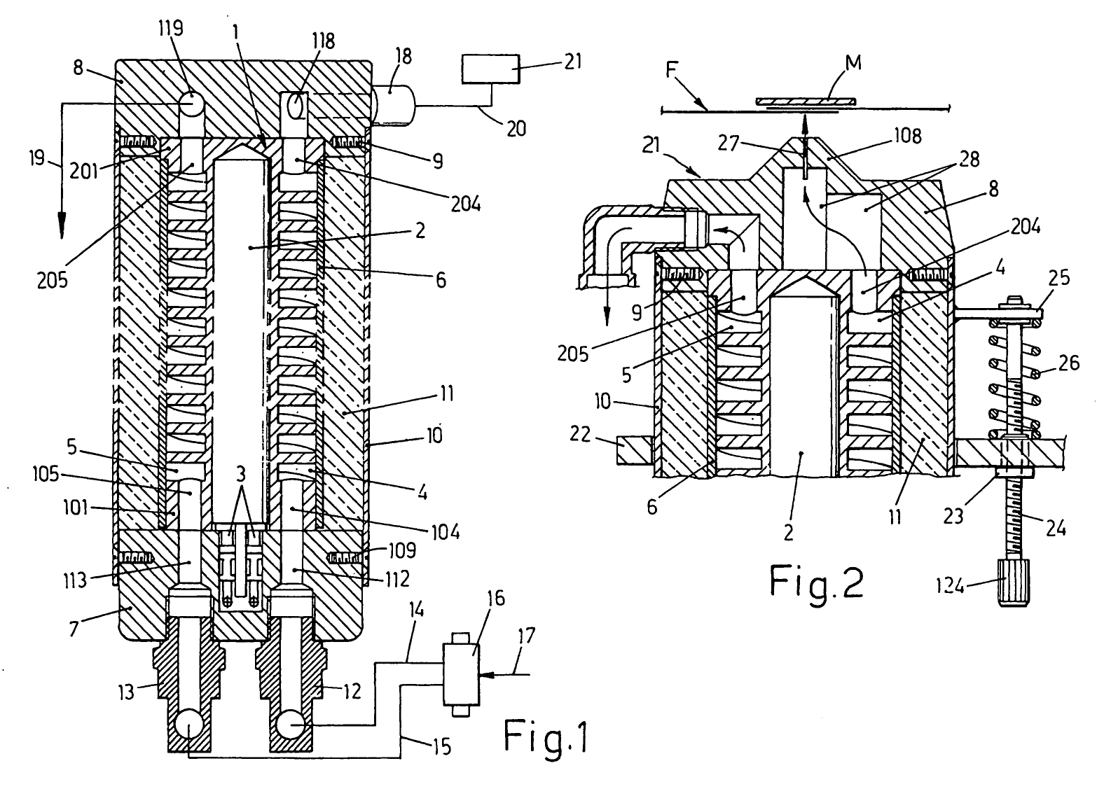

FIG. 1 illustrates the generator of compressed hot air, in section on the longitudinal axis;

FIG. 2 illustrates details of the upper end of the generator of compressed hot air, in the version useful in the construction of a continuous sealer or cutting unit for thermoplastic film.

It can be seen in FIG. 1 that the generator comprises a central body 1 made of for example aluminium or its alloys of cylindrical shape and round cross section with an axial blind cavity 2 housing at least one preferably armoured electrical resistance, housed adjacent to which there are also temperature probes 3 that monitor the operation of the apparatus. Except for small end portions 101 and 201, the rest of the body 1 is machine-recessed on the external lateral surface, in such a way as to be wrapped, like the thread of a two-start screw, by two adjacent channels of cylindrical helical form 4 and 5, having identical dimensional characteristics and having equal coefficients of heat exchange in the direction of the said seat 2 of the electrical resistance. The said channels 4 and 5 communicate via their opposite ends with respective ducts 104, 204 and 105, 205 having identical dimensional characteristics formed in the said end portions 101, 201 of the body 1 and arranged parallel to the axis of this body. The body 1 is covered externally, with slight interference, by a jacket 6 of for example stainless steel or of any other suitable material.

Caps 7 and 8 are connected in intimate contact on the ends of said body 1, the said caps being made of any suitable material with a high degree of heat insulation and for example machinable by machine tools, cylindrically shaped, that are fixed for example by screws 9, 109 or some other suitable method to the ends of a tubular jacket 10 of any suitable material, of a diameter appropriately greater than that of the internal jacket 6, the gap 11 between the two jackets being filled by any suitable material with high characteristics of heat insulation, in such a way that the present apparatus has little or no leakage of heat to the exterior.

The cap 7 contains internal ducts 112 and 113 which on the one hand are connected to the respective ducts 104 and 105 and on the other hand are fixed to the connectors 12 and 13 which, through pipes 14 and 15 and a switching-valve means 16 can be connected alternately and quickly to a pipe 17 that delivers the compressed air at for example room temperature. The opposite cap 8 contains internal ducts 118 and 119 connected to the respective ducts 204 and 205, the duct 119 being connected by a connector to a discharge pipe 19, while the duct 118 is connected through one or more connectors 18 to one or more pipes 20 that supply the working unit 21 of the machine which ejects the compressed hot air and is characterized by cyclical operation.

The apparatus as described works as follows. When the unit 21 is to supply the hot air to the packaging machine, the valve means 16 connect the circuit 12, 112, 104, 4, 204, 118, 18, 20, 21 to the duct 17 supplying air at room temperature. The air at room temperature is heated as it passes through the helical channel 4 by the electrical resistance located inside the seat 2, but without coming into direct contact with this resistance, and passes out at the required temperature via the ducts 204, 118, 18, 20, finally reaching the utilizing appliance 21. The structural and dimensional characteristics of the whole circuit will be such as to guarantee that there is sufficient contact time between the air and the heat source to raise the air to the desired constant temperature. When, on the other hand, the utilizing appliance 21 is not to emit hot air, the valve means 16 are switched at the correct moment to interrupt the supply of compressed air through the pipe 14 so as to divert it to the pipe 15 and along the succeeding circuit 13, 113, 105, 5, 205, 119, 19, which discharges in any area of the packaging machine where the hot air can be freely released or can be employed to improve certain phases of the operating cycle of the packaging machine.

The advantages procured by the solution set out above will be obvious. One advantage is that because the air to be heated does not strike the electrical resistance directly, as in the prior art, but operates in contact with the metal surface of the body 1 and its grooves 4 or 5, then even if it is carrying small drops of water or other impurities, these will not damage the resistance, which as pointed out earlier is preferably shielded, as being more resistant to temperature spikes. A second advantage is due to the fact that the switching-valve means 16 act on the stream of room-temperature air instead of on the stream of hot air, which makes for greater reliability of operation over time of these means because they come under less stress. Another advantage is due to the fact that the two circuits which alternately carry the compressed air and which include the helical channels 4 and 5, can easily be made identical in terms of length, cross-sectional area and more generally in terms of shape and dimensions, in such a way that the amount of heat given off by the resistance to the air is always constant and the working conditions of the electrical resistance are not varied. The operation of the electrical resistance can thus be controlled very safely by the temperature probes 3 (preferably two for redundancy) alone, which cut or throttle the supply to the resistance when they detect temperature values exceeding the set limits. This makes it superfluous to subordinate the operation of the electrical resistance to a pressure sensor which in the prior art checks that there is a correct stream of air through the electrical resistances.

Another important advantage of the disclosed hot-air generator is represented by the complete absence of leakage of heat. It is this that enables the generator to be located statically in the immediate vicinity of the utilizing appliance 21 and of the packaging film or to be actually incorporated into the utilizing appliance, as illustrated for example in FIG. 2 (see below) which in this particular example shows the construction of a hot air knife sealer for making a longitudinal seal along the overlapping edges of the packaging film F, which travels in contact with a part of the mandrel M for tubularising the film. The generator is arranged at right angles to the film F such that the cap 8 with the hot-air outlet ducts are adjacent to it and is guided through the external jacket 10 by fixed vertical guide means 22 to which there is attached, for example, a nut 23 engaging with a vertical screw 24 with an adjusting knob 124 at its bottom end while its top end is connected axially and freely rotatably to a projection 25 integral with the said jacket 10, the whole in such a way that by means of the screw 24 it is possible to precisely adjust the distance between the present apparatus and the film F, depending on the characteristics of this film. A spring 26 may be located between the projection 25 and the nut 23 in order to push the hot-air generator upwards with at least a force approximately equal to the weight of the said generator, in order to facilitate the turning of the adjustment screw 24. Again in FIG. 2, it can be seen that the top cap 8 has a central upwardly tapering protuberance 108 positioned a short distance away from the film to be sealed F and containing a straight vertical slit 27 which communicates with an internal chamber 28 inside the cap 8, the chamber in turn communicating with the hole 204 connected to the channel 4 supplying the utilizing appliance, all in such a way that when this channel carries the compressed air, a hot air knife necessary in the present example for sealing the overlapping edges of the packaging film F emerges from the said slit 27.

It will be understood that instead of one slit 27 there may be two or more adjacent slits, or that instead of this slit with its continuous aperture there may be a row of holes of suitable diameter. Instead of the slit 27 or its replacement openings, there may be a precision-made slit or a hole or nozzle of suitable diameter and the apparatus may be designed with a heating capacity such as to emit a pencil or knife of compressed hot air capable of cutting plastic film or sheets, by keeping the generator stationary or moving it with respect to the surface to be cut, e.g. by means of slides, with a movement system based on Cartesian axes or a robotic arm. In this version the generator may be used for example to make longitudinal separating or opening cuts on the films as they pass out of the production cycle, or to make so-called travelling or shaped cuts, as an alternative to the usual mechanical blanking or cutting operations.

1. Static generator of compressed hot air for delivery to cyclically operated utilizing appliances intended for example for operations of heating, sealing or cutting thermoplastic films or sheets, of the type comprising at least one heat source usually consisting of an electrical resistance controlled by temperature probes (3) to heat a flow of air or other gas under pressure which is forced through the said generator and then directed to the utilizing appliance, characterized in that the electrical resistance is housed in a composite body made of a material which is a good heat conductor, externally insulated so as not to leak heat into the external environment and machined in any way so that it comprises internally at least two separate and offset channels (4, 5) having identical dimensional characteristics and having essentially equal surface areas of contact with the resistance and essentially equal coefficients of heat exchange and that are provided with respective inlet ports (112, 113) and with respective outlet ports (118, 119), the inlet ports being connectable as required through switching-valve means (16) to the source (17) supplying the compressed air for heating, while the two discharge ports of the said channels are connected, one to the utilizing appliance (21), for example to the sealer, to the thermal conditioning station, to the cutting station or to another cyclically operated utilizing appliance and the other discharge port is connected to an exhaust duct (19) that ends in any position remote from the said utilizing appliance, the whole in such a way that by supplying one or other of the said channels with the stream of compressed ambient air, the compressed hot air produced by the generator can be sent to the utilizing appliance or to the said exhaust duct while maintaining unchanged heat exchange conditions between the resistance and the stream of air whatever path the air flows, owing to the structural identity and to the uniformly distributed offset locations of the said channels.

2. Generator according to claim 1, characterized in that the internal channels (4, 5) of the said generator, which alternately carry the stream of air to be heated and which prevent direct contact between the air and the electrical resistance, are shaped as adjacent cylindrical helices, like the threads of a two-start screw.

3. Generator according to claim 2, characterized in that it comprises a central body (1) made of for example aluminium or its alloys of cylindrical shape and round cross section with an axial cavity (2) capable of accommodating at least one preferably armoured electrical resistance, housed adjacent to which there are also temperature probes (3) that monitor the operation of the generator in relation to predetermined temperature limits, the said body (1) being machine-recessed on the external lateral surface, in such a way as to be wrapped, like the thread of a two-start screw, by two adjacent channels of cylindrical helical form (4, 5), having identical dimensional characteristics and having equal surface areas and equal coefficients of heat exchange in the direction of the said seat (2) with the electrical resistance, these channels (4, 5) communicating via their opposite ends with respective holes (104, 204 and 105, 205) having identical dimensional characteristics located on the unmachined end portions (101, 201) of the central body (1) and arranged parallel to the axis of this body, which is covered externally, for example with slight interference, by a tubular jacket (6) of any suitable material, caps (7, 8) being connected in intimate contact on the ends of said central body (1), the said caps being made of any suitable material or alloy of materials with a high degree of heat insulation and for example machinable by machine tools, cylindrically shaped, that are fixed for example by screws (9, 109) or some other suitable method to the ends of a tubular jacket (10) of any suitable material, of a diameter appropriately greater than that of the internal jacket (6), and the gap (11) between the two jackets being occupied by any suitable material with high characteristics of heat insulation, which prevents any leakage of heat to the exterior, one of the said caps (7) containing internal ducts (112, 113) which on the one hand are connected to the respective end ducts (104, 105) of the said helical channels and on the other hand are fixed to the connectors (12, 13) which, through pipes (14, 15) and a switching-valve means (16) can be connected alternately and quickly to a pipe (17) that supplies the generator with the compressed air at for example room temperature, the opposite cap (8) containing internal ducts (118, 119) connected at one end to the respective other end ducts (204, 205) of the said helical channels (4, 5) and at the other end the duct (119) being connected by a connector to a discharge pipe (19), while the duct (118) is connected through one or more connectors (18) to one or more pipes (20) that supply the working unit or utilizing appliance (21) which ejects the compressed hot air and is characterized by cyclical operation.

4. Generator according to claim 3, characterized in that at least the internal tubular jacket (6) surrounding the inner body (1) with the cylindrically helical channels (4, 5) is made for example from stainless steel or other suitable material.

5. Generator according to claim 1, characterized in that because there is no leakage of heat, the said generator can be located statically in the immediate vicinity of the utilizing appliance (21) and of the packaging film which the latter is handling.

6. Generator according to claim 5, characterized in that the said generator may incorporate the utilizing appliance to which it is supplying the hot air.

7. Generator according to claim 6, characterized in that if it is intended for the formation of a sealer that projects at least one hot air knife onto the overlapping edges of the film (F) to be sealed, the said generator is oriented at right angles to the said film edges, the caps (8) with the hot air outlet ducts being next to this film and having a central upwardly tapering protuberance (108) positioned a short distance away from the film to be sealed and containing at least one straight vertical slit (27) formed by a continuous fissure or by a line of holes, which communicates with an internal chamber (28) inside the said cap (8), the chamber in turn communicating with the hole (204) connected to the helical channel (4) supplying the utilizing appliance, all in such a way that when this channel is supplied with the compressed air, the hot air knife useful in the film (F) heat-sealing operation emerges from the said slit (27).

8. Generator according to claim 7, characterized in that it is guided through the external jacket (10) by fixed vertical guide means (22) which directly or indirectly support, and to which there is attached, a nut (23) engaging with a vertical screw (24) with an adjusting knob (124) at its bottom end while its top end is connected axially and freely rotatably to a projection (25) integral for example with the external jacket (10), the whole in such a way that by means of the said screw (24) it is possible to precisely adjust the distance between the present apparatus and the film to be sealed (F), depending on the characteristics of this film, a spring (26) being located between the said projection (25) and the nut (23) in order to push the hot-air generator upwards with at least a force approximately equal to the force of the weight of the said generator, in order to facilitate the turning of the said adjusting screw (24).

9. Generator according to claim 6, characterized in that if it is intended for the formation of a unit for cutting plastic film or sheets and for this purpose projecting an air pencil or knife at the correct pressure and temperature, the said generator is oriented at right angles to the surface to be cut, the cap (8) with the hot air outlet ducts being next to this surface having an upwardly tapering central protuberance (108) positioned a short distance away from the said surface to be cut and containing a precision-made incision or hole or nozzle which communicates with an internal chamber (28) inside the said cap (8), this chamber in turn communicating with the hole (204) connected to the helical channel (4) supplying the utilizing appliance, all in such a way that when this channel is supplied with compressed air, the hot compressed air knife or pencil useful for cutting the nearby plastic sheet or film emerges from the said precision-made incision, hole or nozzle.

10. Generator according to claim 9, characterized in that it can be used statically for making in-line cuts or in that it can be mounted on simple or complex movement means for making travelling or shaped cuts.
